(19) United States
(12) Patent Application Publication
(10) Pub. No.: US 2010/0286283 A1
(43) Pub. Date: Nov. 11, 2010

(54) MICROTUBULE-DISRUPTNG AGENT AND CANCER CELL PROLIFERATION INHIBITOR CONTAINING THE SAME

(76) Inventors: Kazuhiro Ishiguro, Nagoya-shi (JP); Hidemi Goto, Nagoya-shi (JP); Takafumi Ando, Nagoya-shi (JP)

Correspondence Address: EDWARDS ANGELL PALMER & DODGE LLP, P.O. BOX 55874, BOSTON, MA, 02205, US

(21) Appl. No.: 12/599,830

(22) PCT Filed: May 1, 2009
(86) PCT No.: PCT/JP2008/058354

§ 371 Date: Jul. 19, 2010

(30) Foreign Application Priority Data
May 15, 2007 (JP) 2007-128878

(51) Int. Cl. A61K 31/12; A61K 31/121; A61P 35/00

(52) U.S. Cl. 514/678; 514/675; 568/308; 568/417

(57) ABSTRACT

The present invention provides a novel microtubule-disrupting agent, and also provides a use of the microtubule-disrupting agent. A microtubule-disrupting agent containing an α,β-unsaturated carbonyl compound as an active ingredient is provided. Further, a cancer cell proliferation inhibitor containing the microtubule-disrupting agent is also provided. As the α,β-unsaturated carbonyl compound, 6-shogaol is preferably used.

### TECHNICAL FIELD

The present invention relates to a microtubule-disrupting agent and a use thereof.

### BACKGROUND ART

Gastric cancer was once at the top cause of cancer death in Japan and currently also ranks among the highest causes. Development of the endoscopic technology made it possible to discover gastric cancer early and excise it with an endoscope; however, an operation is impossible for advanced gastric cancer that has already developed metastasis, and in this case, chemotherapy is mainly performed.

In chemotherapy to inoperable advanced gastric cancer, a certain degree of a survival advantage can be expected, but such a survival advantage is not shown in all cases, and even in cases showing survival advantages, resistance and the like develop and most of the cases reach relapse.

In order to increase cases where chemotherapy is effective (expansion of adaptive cases) and avoid resistance and extend life-prolonging span to further attain curing (improvement of effect), it is required to newly identify an antitumor agent effective for cancer treatment. The new antitumor agent is used alone or combined with conventional antitumor agents, which makes it possible to form a new protocol of chemotherapy.

Documents relating to 6-shogaol (patent documents) will be listed below. Patent Document 1 discloses a method for extracting 6-shogaol and refers to applications of shogaol (anti-inflammatory pharmaceutical composition, anti-platelet aggregation pharmaceutical composition, and animistic pharmaceutical composition). Patent Document 2 refers to applications of shogaol (dyspepsia therapeutic agent, antiemetic agent, antidiabetic drug, painkiller, antirheumatic drug, and nutritious supplement). Patent Document 3 also refers to applications of shogaol (fragrance, skin external preparation, febrifuge, painkiller, anti-inflammatory agent, cough suppressant, and antioxidant).

Furthermore, synthesis of shogaol analogous compounds (not containing 6-shogaol) has been reported (Patent Document 4). In Patent Document 4, effects for gene expressions depending on transcription factor Nrf2 are evaluated on various shogaol analogous compounds. However, the evaluation is only based on a level of mRNA, not a level of protein. In addition, even though foods, medical products, and quasi drugs are shown as application fields of the compounds disclosed there, they are not supported by experimental data and specific applications cannot be grasped.

### [Patent Document 1] Japanese Unexamined Patent Publication (JP-A) No. 2000-047195

### [Patent Document 2] National Publication of International Patent Application No. 2005-511641

### [Patent Document 3] JP-A No. 2003-327574

### [Patent Document 4] JP-A No. 2006-188444

### DISCLOSURE OF THE INVENTION

### Problems to be Solved by the Invention

An object of the present invention is to provide a novel microtubule-disrupting agent. Another object thereof is to provide a use of the microtubule-disrupting agent.

### Means for Solving the Problems

As a result of intensive studies in view of the above-described problems, the present inventors discovered from cell-based experiments and animal experiments that 6-shogaol, which is one component contained in ginger, disrupted microtubules of various cancer cells, thereby stopping cell divisions, and that cell viability and proliferation were significantly inhibited. When the inventors proceeded further studies focusing on the structure characteristic to 6-shogaol, they achieved such a finding that “an α,β-unsaturated carbonyl compound having a chemical structure in common with 6-shogaol reacted with an SH group in a tubulin and inhibited polymerization of the tubulin, thereby disrupting a structure of microtubule”.

The present invention is based on the above-described achievement, and provides the following microtubule-disrupting agent, cancer cell proliferation inhibitor, and the like.

[1] A microtubule-disrupting agent containing an α,β-unsaturated carbonyl compound having a chemical structure in common with 6-shogaol as an active ingredient.
[2] The microtubule-disrupting agent according to [1], wherein the compound is a compound defined by the following chemical formula:

R₁—R₂—CO—CH═CH—R₃ [Formula 1]

wherein R₁ is an aryl group having 6 to 18 carbon atoms, R₂ is an alkyl group having 1 to 10 carbon atoms, and R₃ is an alkyl group having 1 to 10 carbon atoms.

[3] The microtubule-disrupting agent according to [1], wherein the compound is a compound defined by the following chemical formula:

R₁—CO—CH═CH—R₂ [Formula 2]

wherein R₁ is an alkyl group having 1 to 10 carbon atoms, and R₂ is an alkyl group having 1 to 10 carbon atoms.

[4] The microtubule-disrupting agent according to [1], wherein the compound is a compound defined by the following chemical formula (6-shogaol):

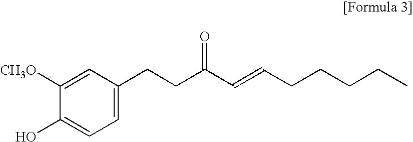

[5] The microtubule-disrupting agent according to [1], wherein the compound is a compound defined by any one of the following chemical formulas:

CH₃—CO—CH═CH—(CH₂)₅—CH₃

CH₃—CO—CH═CH—(CH₂)₄—CH₃

CH₃—CO—CH═CH—(CH₂)₃—CH₃

CH₃—CO—CH═CH—(CH₂)₂—CH₃ [Formula 4]

[6] A cancer cell proliferation inhibitor containing a microtubule-disrupting agent according to any one of [1] to [5].
[7] The cancer cell proliferation inhibitor according to [6], which is used for prevention or treatment of cancer.
[8] The cancer cell proliferation inhibitor according to [7], wherein the cancer is selected from the group consisting of gastric cancer, acute T-cell leukemia, and colon cancer.
[9] A method for prevention or treatment of cancer, wherein the cancer cell proliferation inhibitor according to [6] is administered to a subject.
[10] A use of a compound according to any one of [1] to [5] for producing a cancer cell proliferation inhibitor.

### BRIEF DESCRIPTION OF DRAWINGS

FIG. 1 is a graph showing an effect of 6-shogaol on gastric cancer cells (HGC-27 cells, AGS cells, and KATO III cells). The horizontal axis indicates a concentration of addition of 6-shogaol, and the vertical axis indicates cell viability. White circle: HGC-27 cells, black circle: AGS cells, gray circle: KATO III cells.

FIG. 2 is a graph showing an effect of 6-shogaol on gastric cancer cells transplanted to a nude mouse. The horizontal axis indicates an administration concentration of 6-shogaol, and the vertical axis indicates a growth ratio of tumor.

FIG. 3 is views showing influences of 6-shogaol given to actin and a microtubule of the gastric cancer cells (result of immunostaining). Left column: phase contrast image, right column: image of immunostaining.

FIG. 4 is a table showing an influence of 6-shogaol given to a cell cycle. Gastric cancer cells were incubated in the presence of shogaol (2 μg/ml) for 18 hours, and then, % of cells in the G2-M stage (n=3) was shown.

FIG. 5 shows an effect of 6-shogaol (24 hour-culture) to viable ratios of various cancer cells. ED50: concentration of 6-shogaol, which decreases the cell viability to 0.5 (50%) as compared with the control.

FIG. 6 shows a result of immunostaining of tubulins constituting a microtubule. Left column: phase contrast image, right column: immunostaining image. The scale bar in the figure indicates 10 μm:

FIG. 7 shows an influence given to polymerization of tubulins (result of tubulin polymerization experiment). Polymerization of 20 μM of tubulins was evaluated by measuring an absorbance at a wavelength of 340 nm. As compared with the control (∘), CH₃—CO—CH═CH—(CH₂)₄—CH₃ (250 μM: , 500 μM: ▪) inhibited polymerization of tubulins, but CH₃—CO—(CH₂)₆—CH₃ (500 μM: Δ) and CH₃—CH₂—CH═CH—(CH₂)₄—CH₃ (500 μM: x) did not give an influence.

FIG. 8 shows an influence given to an SH group of a tubulin necessary for polymerization (result of experiment of SH group quantitative determination). Tubulins (2 μM) and CH₃—CO—CH═CH—(CH₂)₅—CH₃, CH₃—CO—CH═CH—(CH₂)₄—CH₃, CH₃—CO—CH═CH—(CH₂)₃—CH₃ or CH₃—CO—CH═CH—(CH₂)₂—CH₃ (500 μM) were reacted, and then, an amount of SH groups contained in the tubulins was evaluated in the Ellman method. An α,β-unsaturated carbonyl compound having a larger molecular weight was reacted with more SH groups of the tubulin to decrease the amount of the SH groups.

FIG. 9 shows binding with a tubulin (result of tubulin binding experiment). A tubulin and a fluorescence dye NBD-PZ or NBD-PZ-CO—CH═CH—(CH₂)₅—CH₃ were reacted, and then electrophoresis (nonreducing SDS-PAGE) was performed. NBD-PZ-CO—CH═CH—(CH₂)₅—CH₃ was bonded with the tubulin, and as a result, comigrated with the tubulin (third lane). When 6-shogaol is coexisted, it binds to the tubulin in place of NBD-PZ-CO—CH═CH—(CH₂)₅—CH₃, and thus, comigration was inhibited (fourth lane). 6-gingerol, which do not have a —CO—CH═CH— structure, did not give an influence to comigration. These facts suggested that a structure of —CO—CH═CH— is important for binding with a tubulin.

FIG. 10 shows an influence given to a viable cell ratio of HGC cells. HGC-27 cells were incubated with CH₃—CO—CH═CH—(CH₂)₄—CH₃, CH₃—CO—(CH₂)₆—CH₃ or CH₃—CH₂—CH═CH—(CH₂)₄—CH₃ (0 to 40 μM) for 24 hours, and then, the cell viability thereof was evaluated. Only CH₃—CO—CH═CH—(CH₂)₄—CH₃ (∘) decreased the cell viability.

### BEST MODE FOR CARRYING OUT THE INVENTION

### 1. Microtubule-Disrupting Agent

A first aspect of the present invention provides a microtubule-disrupting agent containing an α,β-unsaturated carbonyl compound having a chemical structure in common with 6-shogaol as an active ingredient. The α,β-unsaturated carbonyl compound means a compound having —CO—CH═CH—.

The “α,β-unsaturated carbonyl compound having a chemical structure in common with 6-shogaol” is defined by either of the following chemical formulas (Formula 5 or 6). In addition, 6-shogaol itself falls into the “α,β-unsaturated carbonyl compound having a chemical structure in common with 6-shogaol”.

R₁—R₂—CO—CH═CH—R₃ [Formula 5]

Provided that R₁ is an aryl group having 6 to 18 carbon atoms, R₂ is an alkyl group having 1 to 10 carbon atoms, and R₃ is an alkyl group having 1 to 10 carbon atoms.

R₁—CO—CH═CH—R₂ [Formula 6]

Provided that R₁ is an alkyl group having 1 to 10 carbon atoms, and R₂ is an alkyl group having 1 to 10 carbon atoms.

A specific example of the compound defined by the chemical formula (Formula 5) is 6-shogaol. The chemical formula of 6-shogaol is shown below. As shown in the section of Examples, 6-shogaol has a particularly strong action of-disrupting a microtubule. Thus, 6-shogaol is preferably used as an active ingredient.

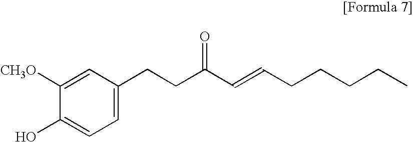

6-shogaol can be prepared by a known method. For example, 6-shogaol can be obtained by extraction and purification from ginger, which is a zingiberaceous plant (for example, see JP-A No. 6-183959). Furthermore, methods for preparing shogaol by chemical synthesis were also developed (for example, see JP-A No. 61-137834, JP-A No. 8-40970, and JP-A No. 2003-327574), and shogaol may be obtained by these methods.

The microtubule-disrupting agent of the present invention may be constituted with not only highly purified 6-shogaol, but also 6-shogaol with various purification degrees. That is, the microtubule-disrupting agent of the present invention can also be produced using a ginger extract or 6-shogaol in a middle stage of purification.

Specific examples of the compound defined by the above chemical formula (Formula 6) include the following four compounds:

CH₃—CO—CH═CH—(CH₂)₅—CH₃

CH₃—CO—CH═CH—(CH₂)₄—CH₃

CH₃—CO—CH═CH—(CH₂)₃—CH₃

CH₃—CO—CH═CH—(CH₂)₂—CH₃ [Formula 8]

As a result of studies made by the present inventors, it was revealed that a compound with a larger molecular weight exerted a stronger action. The compound on the first line among the above four compounds showed the strongest action.

A microtubule is a tubular structure widely existing in eukaryotic cells, determinates a distribution of a cell minute organ (organelle) and speculates a cell from, and also functions as a rail for intracellular transport. Further, a microtubule constitutes a spindle fiber in mitotic division, and serves as a central function in cell division. The microtubule-disrupting agent of the present invention exerts an action of disrupting a microtubule serving as such an important function in a cell (that is, an action of inhibiting normal formation).

### 2. Applications of Microtubule-Disrupting Agent

### (1) Inhibition of Proliferation of Cancer Cells

A second aspect of the present invention provides applications of the above-described microtubule-disrupting agent. The first application is inhibition of proliferation of cancer cells. That is, the present invention provides a cancer cell proliferation inhibitor containing the microtubule-disrupting agent. The cancer cell proliferation inhibitor of the present invention is applied to cancer cells or a tissue containing cancer cells. When the cancer cell proliferation inhibitor of the present invention is applied, the “cancer cells or a tissue containing cancer cells” may be in a state of existing in a living body, or may be in a state of being extracted from the living body. Kinds, derivations, malignancy and the like of cancer cells are not particularly limited. Examples of the cancer cells include cancer cells forming various cancers described later. Cancer cells in the present invention are preferably any of gastric cancer cells, malignant T-cells, and colon cancer cells.

Since the cancer proliferation inhibitor of the present invention can inhibit proliferation of cancer cells, it can be used for prevention or treatment of cancers. Vinca alkaloid has been known as an antitumor agent exerting an antitumor action by disrupting a microtubule, but an α,β-unsaturated carbonyl compound being an active ingredient of the cancer proliferation inhibitor of the present invention has a structure completely different from that of vinca alkaloid. Therefore, according to the cancer proliferation inhibitor of the present invention, it is expected to exert effects also to cases where vinca alkaloid is not effective.

The term “cancer” referred in the present invention has the same meaning as a malignant tumor and includes both carcinoma and sarcoma. Herein examples of the cancer include esophageal cancer, oral cavity cancer, maxillary cancer, laryngeal cancer, pharyngeal cancer, gastric cancer, duodenal cancer, colon cancer, liver cell carcinoma, cholangiocellular carcinoma, lung cancer, prostate cancer, renal cancer, bladder papilloma, prostate cancer, urethral epidermoid cancer, osteosarcoma, chondrosarcoma, synovial sarcoma, myxosarcoma, liposarcoma, multiple myeloma, malignant lymphoma, squamous cell carcinoma, malignant melanoma (melanoma), glial tumor, meningioma, neuroblastoma, breast cancer, mammary sarcoma, carcinoma in situ of uterine, carcinoma in situ of uterine cervix, uterine adenocarcinoma, uterine sarcoma, ovarian carcinoma, malignant melanoma (melanoma), thyroid papillary carcinoma, follicular carcinoma of thyroid, acute myeloid leukemia, acute promyelocytic leukemia, acute myelomonocytic leukemia, acute monocytic leukemia, acute lymphatic leukaemia, acute undifferentiated leukemia, chronic myeloid leukemia, chronic lymphoid leukemia, and adult T-cell leukemia. The cancer proliferation inhibitor of the present invention is preferably used in prevention or treatment of gastric cancer, acute T-cell leukemia, or colon cancer.

Preparation of the Cancer Cell Proliferation Inhibitor of the Present Invention can be carried our according to a conventional method. For preparation, other pharmaceutically acceptable components (e.g., carriers, vehicles, disintegrating agents, buffers, emulsifiers, suspending agents, soothing agents, stabilizers, preservatives, antiseptics, and physiological saline) can be contained. As a vehicle, lactose, starch, sorbitol, D-mannitol, sucrose, or the like can be used. As a disintegrating agent, starch, carboxymethyl cellulose, calcium carbonate, or the like can be used. As a buffer, phosphate, citrate, acetate, or the like can be used. As an emulsifier, gum arabic, sodium alginate, tragacanth, or the like can be used. As a suspending agent, glyceryl monostearate, aluminum monostearate, methylcellulose, carboxymethyl cellulose, hydroxymethyl cellulose, sodium lauryl sulfate, or the like can be used. As a soothing agent, benzyl alcohol, chlorobutanol, sorbitol, or the like can be used. As a stabilizer, propylene glycol, diethylin sulfite, ascorbic acid, or the like can be used. As a preservative, phenol, benzalkonium chloride, benzyl alcohol, chlorobutanol, methylparaben, or the like can be used. As an antiseptic, benzalkonium chloride, p-hydroxybenzoic acid, chlorobutanol, or the like can be used.

A dosage form is also not particular limited. Examples of the dosage form include tablets, powders, fine granules, granules, capsules, syrups, injections, external preparations, and suppositories.

In the cancer cell proliferation inhibitor of the present invention, an active ingredient is contained in a necessary amount (i.e., therapeutic effective dose) for obtaining an expected therapeutic effect (or prevention effect). Although an amount of the active gradient in the drug of the present invention is generally different depending on a dosage form, the amount of the active ingredient is set within the range, for example, from about 0.1% by weight to about 95% by weight in order to achieve a desired dose.

The cancer cell proliferation inhibitor of the present invention is applied to a subject by oral administration or parenteral administration (intravenous, intraarterial, subcutaneous, intramuscular or intraperitoneal injection, or transdermal, transnasal, transmucosal, etc.) according to a dosage form thereof. The “subject” referred herein is not particularly limited, and includes human and mammals other than human (including pet animals, livestock, and experimental animals, and specific examples include a mouse, rat, guinea pig, hamster, monkey, cattle, pig, goat, sheep, dog, cat, chicken, and quail). In one preferable embodiment, the cancer cell proliferation inhibitor of the present invention is applied to a human.

A dose of the cancer cell proliferation inhibitor of the present invention is set so as to obtain an expected therapeutic effect. A symptom, age, a sex, a body weight, etc. of a patient are generally taken into consideration for setting a therapeutically effective dose. A person skilled in the art is capable of setting a suitable dose considering these matters. For example, a dose can be set so that an active ingredient amount per day for an adult (body weight of about 60 kg) as a subject is about 50 to about 250 mg, and preferably about 100 mg to about 200 mg. As an administration schedule, for example, once to several times per day, once per two days, or once per three days, and the like, can be employed. A symptom of a patient, a duration of effects of an active ingredient, and the like, can be considered for preparing an administration schedule.

### (2) Breeding

The microtubule disrupting agent of the present invention can be used also in breeding of plants. That is, similar to colchicine that is a known microtubule-disrupting agent, the microtubule-disrupting agent of the present invention may be used to produce polyploids and excellent breeds of fruits (citrus, berries, etc.) and vegetables (lettuce, etc.). A treatment method when the microtubule-disrupting agent of the present invention is used in such an application may be followed in accordance with a treatment method with colchicines. Examples of the treatment method include a method for immersing a seed or a germinating seed in the microtubule-disrupting agent or a dissolved solution thereof, and a method for applying or spraying the microtubule-disrupting agent or a dissolved solution thereof to a seedling, a young tree, or the like. An amount of an active ingredient (α,β-unsaturated carbonyl compound such as 6-shogaol) in a treating solution used herein, a treatment time, etc. are different depending on an object for the treatment and a subject to be treated, and suitable conditions can be set through preliminary experiments.

### EXAMPLES

### 1. Effect of 6-Shogaol on Gastric Cancer Cells

1×10⁴ gastric cancer cells (HGC-27 cells, AGS cells, and KATO III cells) were seeded in 96 well-plates, and incubated with 6-shogaol (0 to 4 μg/ml) for 24 hours from the next day. Viability of the cells was evaluated in a coloring method using Cell Titer 96 cell proliferation assay (Promega Co., Madison, Wis.), and an absorbance was measured. In this assay, living cells develop color with a reagent, and as the number of living cells is larger, the absorbance is higher. The cell viability was calculated by (absorbance at each addition concentration of 6-shogaol)/(absorbance at 0 μg/ml). Therefore, the viable cell ratio was 1.0 on the basis of 0 μg/ml. 6-shogaol was purchased from Wako Pure Chemical Industries, Ltd. (Osaka, Japan) (purity of 98% or more). Each gastric cancer cell was available from the cell bank in RIKEN BioResource Center (RIKEN BRC cell bank) or ATCC (American Type Culture Collection).

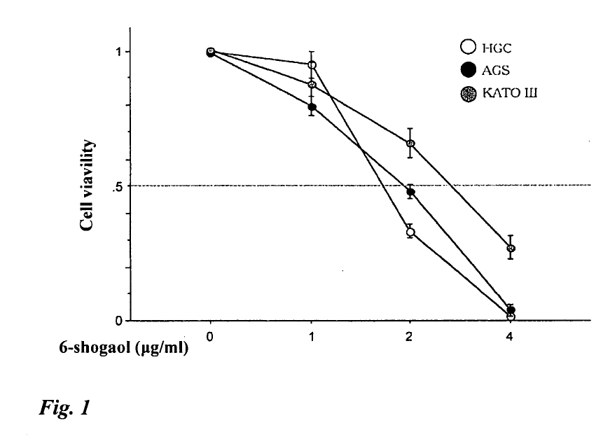

As shown in FIG. 1, 6-shogaol decreased the cell viability of all gastric cancer cell lines.

### 2. Effect of 6-Shogaol on Tumor

2.5×10⁶ gastric cancer cells (HGC-27 cells) were subcutaneously inoculated into the flank of a nude mouse (7-week-old, purchased from Japan SLC, Inc.). After 3 weeks when the tumors had reached an average volume of approximately 40 mm³, and then 400 μl of 6-shogaol at 0 to 200 μg/ml was administered into the abdominal cavity for 8 days. The tumor volume was measured before the first administration of 6-shogaol and after one day from the eighth administration. Comparing volumes of the tumor before and after administration of 6-shogaol, its magnification was evaluated as a growth ratio of the tumor.

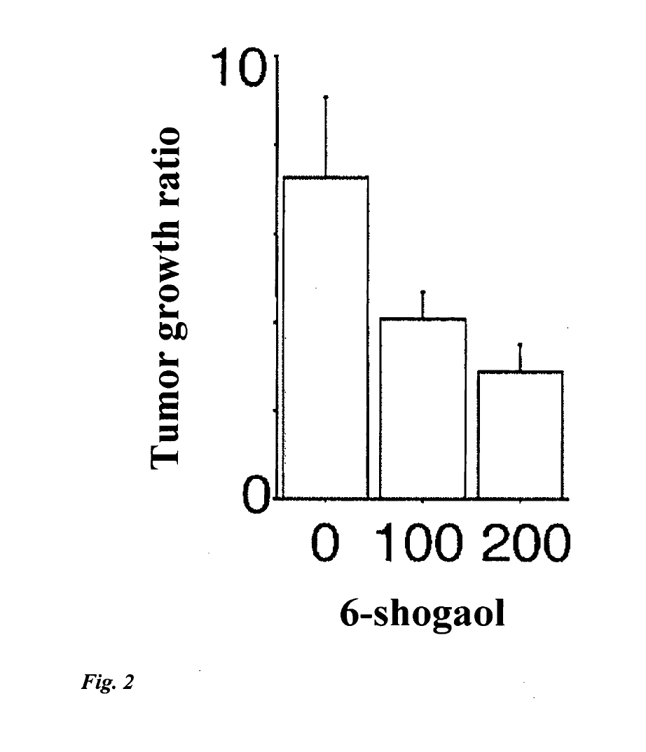

As shown in FIG. 2, the growth of the tumor was inhibited by 6-shogaol.

### 3. Action Mechanism of 6-Shogaol

### (1) Immunostaining Experiment

Gastric cancer cells (HGC-27 cells) were incubated with 6-shogaol at 2 μg/ml for 6 hours and then fixed, and actin was fluorescent-stained with Alexa Fluor phalloidin (Molecular Probes Inc., Eugene, Oreg.), and tubulins were fluorescent-stained with an anti-tubulin β antibody (Lab Vision Co., Fremont, Calif.) and Alexa Fluor 488 labeled second antibody (Molecular Probes, Inc.). The cells were observed with a fluorescent microscope manufactured by Keyence Corporation (Tokyo, Japan).

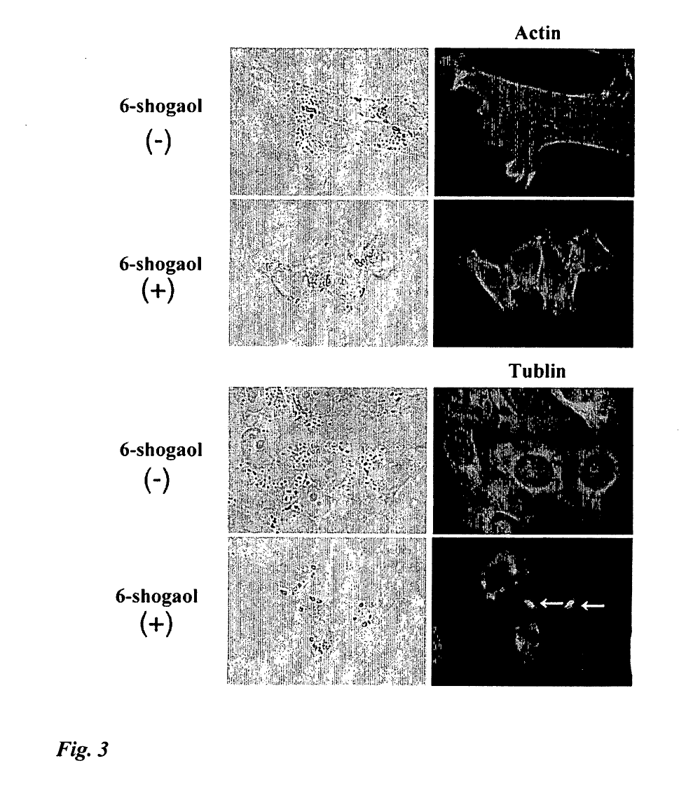

As shown in FIG. 3, when incubated with 6-shogaol for 6 hours, a distribution of actin (cortical distribution) was not affected, but a distribution of tubulins constituting a microtubule (reticulate distribution) was disrupted and coagulation of the tubulins was observed. This result suggested that 6-shogaol had an action of disrupting microtubules.

### (2) Influence Given to Cell Cycle

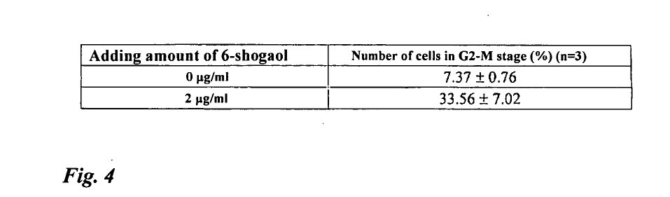

2×10⁵ gastric cancer cells (HGC-27 cells) were seeded in 12-well plates and incubated in the presence of 6-shogaol at 2 μg/ml for 18 hours from the next day, and then, further incubated in the presence of 10 μM of BrdU for 2 hours. In order to measure intake of BrdU in nuclei and DNA amounts in the nucleus, the cells were stained using a BrdU flow kit (BD Pharmingen, Inc., San Diego, Calif.). 10000 cells were analyzed with the flow cytometer manufactured by Beckman Coulter, Inc. (Fullerton, Calif.). The cells containing a large amount of DNA and a small amount of BrdU were considered to be in G2-M phase. As shown in FIG. 4, it was suggested that adding 6-shogaol significantly increased a ratio of cells in G2-M stage and cell divisions of gastric cancer cells were stopped due to disruption of microtubules.

### 4. Effects of 6-Shogaol on Various Cancer Cells

ED50 of 6-shogaol was examined on various cancer cells (HGC-27 cells, AGS cells, KATO III cells, Jurkat cells, A549 cells, and SW620 cells) to evaluate whether or not 6-shogaol was also effective for cancer cells other than gastric cancer cells. The culturing time was set to 24 hours.

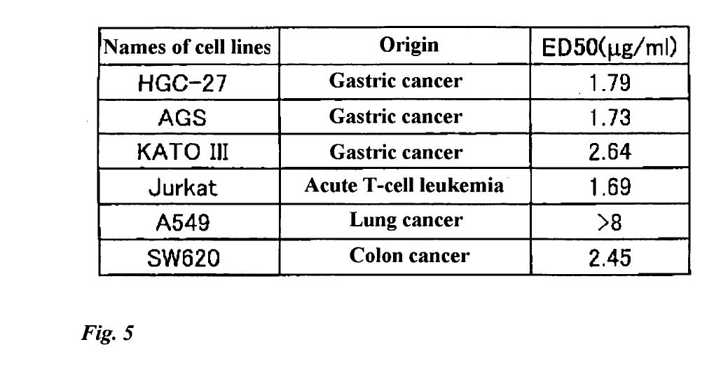

As shown in FIG. 5, 6-shogaol decreased the viability in all of the cancer cells. However, susceptibility to 6-shogaol was low in A549 cells (liver cancer cells) as compared with the other cancer cells.

### 5. Summary

As described above, it was shown that 6-shogaol inhibited proliferation of cancer cells. What is more, it was revealed that as its action mechanism, microtubules were disrupted, which accompanied prevention of cell division.

### 6. Immunostaining Experiment

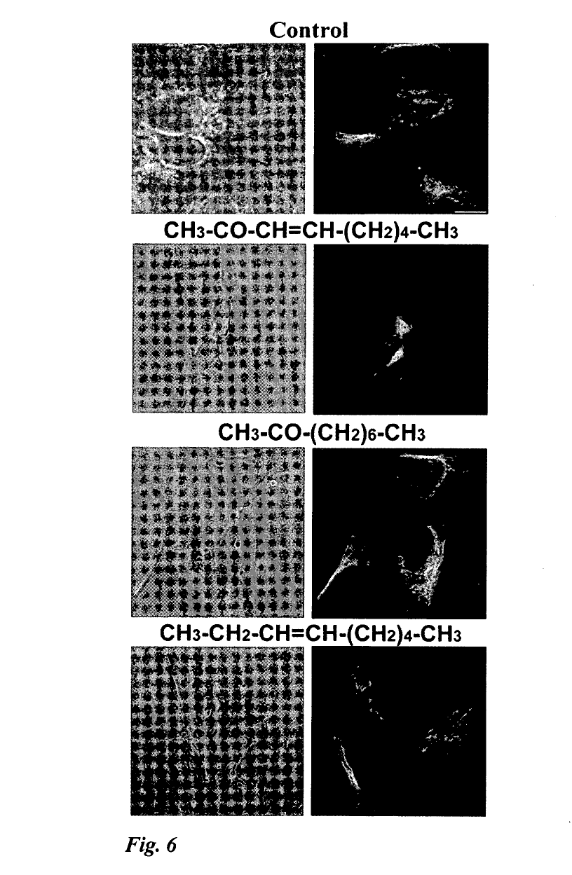

Gastric cancer cells (HGC-27 cells) were incubated with CH₃—CO—CH═CH—(CH₂)₄—CH₃, CH₃—CO—(CH₂)₆—CH₃ or CH₃—CH₂—CH═CH—(CH₂)₄—CH₃ (40 μM) for 6 hours and then fixed, and tubulins were fluorescent-stained with anti-tubulin β antibody (Lab Vision Co., Fremont, Calif.) and Alexa Fluor 488 labeling second antibody (Molecular Probes, Inc.). The cells were observed with the fluorescent microscope manufactured by Keyence Corporation (Tokyo, Japan). The result suggested that only CH₃—CO—CH═CH—(CH₂)₄—CH₃ inhibited a reticulate distribution of tubulins and a structure of a microtubule was disrupted (FIG. 6).

### 7. Experiment of Tubulin Polymerization

Along with gradually polymerizing tubulins in a polymerization solution (80 mM PIPES, pH 6.9, 2 mM MgCl₂, 0.5 mM EGTA, 5% glycerol and 1 mM GTP), an absorbance of the solution at a wavelength of 340 nm increases. The absorbance (340 nm) was measured in every 5 minutes to thus evaluate an influence given to the polymerization.

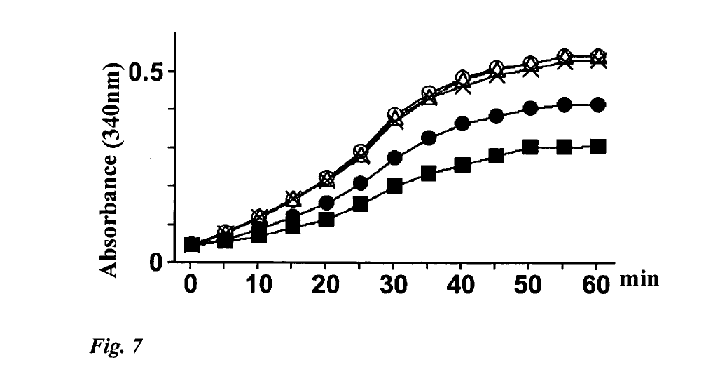

CH₃—CO—CH═CH—(CH₂)₄—CH₃ (250 μM: , 500 μM: ▪) inhibited polymerization of tubulins as compared with the control (∘), but CH₃—CO—(CH₂)₆—CH₃ (500 μM: Δ) and CH₃—CH₂—CH═CH—(CH₂)₄—CH₃ (500 μM: x) gave no influence (FIG. 7).

### 8. Quantification of SH Group

SH groups contained in tubulins are converted to 5,5′-dithiobis-nitrobenzoic acid into thionitrobenzoate, and an absorbance of the solution at a wavelength of 412 nm (Ellman reaction) increases. The absorbance (412 nm) was measured to evaluate an influence given to an amount of SH groups.

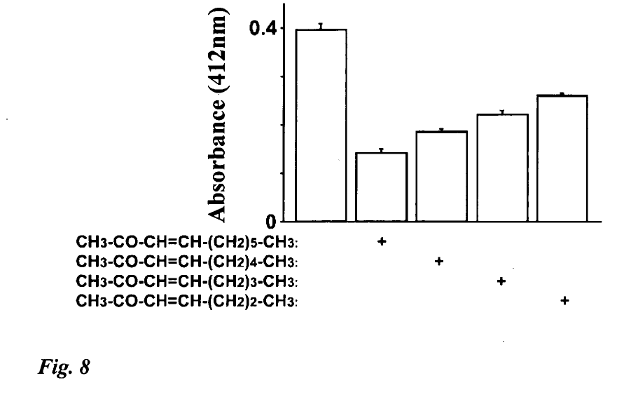

Tubulins (2 μM) and CH₃—CO—CH═CH—(CH₂)₅—CH₃, CH₃—CO—CH═CH—(CH₂)₄—CH₃, CH₃—CO—CH═CH—(CH₂)₃—CH₃ or CH₃—CO—CH═CH—(CH₂)₂—CH₃ (500 μM) were reacted, and then, an amount of SH groups contained in the tubulins was evaluated with the Ellman method. An α,β-unsaturated carbonyl compound having a larger molecular weight reacted with more SH groups in the tubulins, and as a result, the amount of the SH groups was decreased and the absorbance (412 nm) was lowered (FIG. 8). In addition, 100 μM of 6-shogaol showed approximately the same effect as 500 μM of CH₃—CO—CH═CH—(CH₂)₅—CH₃.

### 9. Tubulin Binding Experiment

Tubulins and a fluorescence dye, NBD-PZ or NBD-PZ-CO—CH═CH—(CH₂)₅—CH₃ were reacted and electrophoresis (SDS-PAGE) was then performed. Binding with the tubulins was determined with comigration (fluorescence derived from NBD-PZ was detected with tubulins).

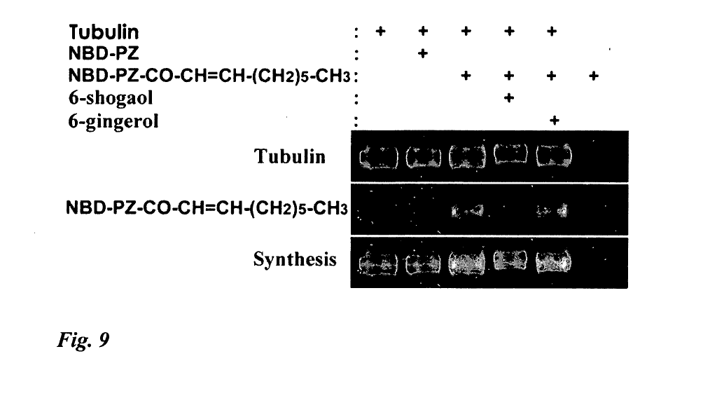

NBD-PZ-CO—CH═CH—(CH₂)₅—CH₃ was bonded with the tubulins, and as a result, comigrated with the tubulins (FIG. 9, third lane). When 6-shogaol coexisted, it was bonded with the tubulins in place of NBD-PZ-CO—CH═CH—(CH₂)₅—CH₃, and thus, the comigration was inhibited (FIG. 9, fourth lane). 6-gingerol that does not have a —CO—CH═CH— structure did not give an influence on comigration. These facts suggested that a structure of —CO—CH═CH— was important for binding with tubulins.

### 10. Effect on Viable Cell Ratio of HGC-27 Cells

1×10⁴ gastric cancer cells (HGC-27 cells) were seeded in 96-well plates, and incubated with CH₃—CO—CH═CH—(CH₂)₄—CH₃, CH₃—CO—(CH₂)₆—CH₃ or CH₃—CH₂—CH═CH—(CH₂)₄—CH₃ (0 to 40 μM) for 24 hours from the next day. Cell viability was evaluated in a coloring method using CellTiter96 cell proliferation assay (Promega Co., Madison, Wis.), and an absorbance was measured. In this assay, living cells develop color with a reagent, and as the number of living cells is larger, the absorbance is higher. The viable cell ratio was calculated by (absorbance at each addition concentration)/(absorbance at 0 μM). Therefore, the viable cell ratio was 1.0 on the basis of 0 μM.

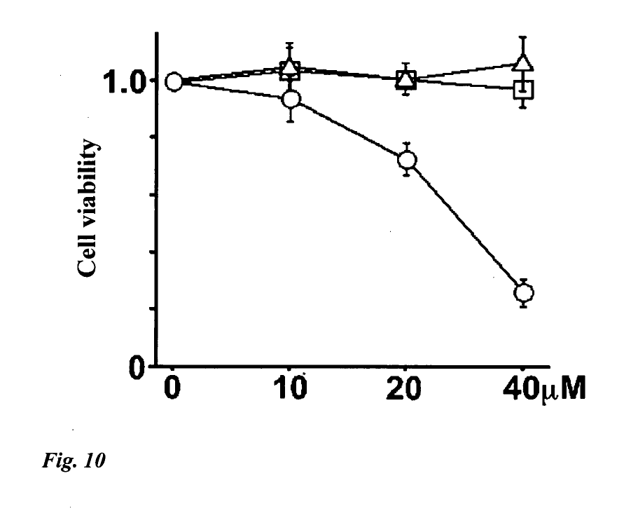

As shown in FIG. 10, only CH₃—CO—CH═CH—(CH₂)₄—CH₃ (∘) decreased the viable cell ratio.

### 11. Conclusion

The above-described results suggested that an α,β-unsaturated carbonyl compound such as 6-shogaol reacted with SH groups in tubulins and inhibited polymerization of the tubulins, thereby disrupting a structure of microtubule.

### INDUSTRIAL APPLICABILITY

The present invention can be used for inhibition of proliferation of cancer cells. An α,β-unsaturated carbonyl compound that is the active ingredient of the present invention has a molecular structure completely different from vinca alkaloid which is known to exerts an antitumor action by disrupting microtubules. Therefore, according to the present invention, it is expected to exhibit a pharmaceutical effect also to cancer cases where vinca alkaloid is not effective.

On the other hand, the present invention can also be intended for its application in the field of breeding (production of polyploid and excellent breed).

The present invention is not limited to the above-described embodiments and examples at all. Various modifications are also included in the invention within the range where these modifications do not depart from the scope of claims and can be easily conceived by a person skilled in the art.

Contents of treatises, unexamined patent publication bulletins, and examined patent publication bulletins are all incorporated herewith by their references.

1. A method for prevention or treatment of cancer, wherein the cancer cell proliferation inhibitor comprising:
a microtubule-disrupting agent consisting of an α,β-unsaturated carbonyl compound having a chemical structure in common with 6-shogaol as an active ingredient.

2. The method according to claim 1, wherein the compound is a compound defined by the following chemical formula:
R₁—R₂—CO—CH═CH—R₃ [Formula 1]
wherein R₁ is an aryl group having 6 to 18 carbon atoms, R₂ is an alkyl group having 1 to 10 carbon atoms, and R₃ is an alkyl group having 1 to 10 carbon atoms.

3. The method according to claim 1, wherein the compound is a compound defined by the following chemical formula:
R₁—CO—CH═CH—R₂ [Formula 2]
wherein R₁ is an alkyl group having 1 to 10 carbon atoms, and R₂ is an alkyl group having 1 to 10 carbon atoms.

4. The method according to claim 1, wherein the compound is a compound defined by the following chemical formula (6-shogaol):
[Formula 3]

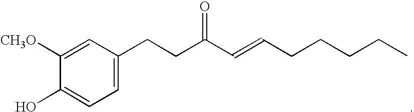

5. The method according to claim 1, wherein the compound is a compound defined by any one of the following chemical formulas:
CH₃—CO—CH═CH—(CH₂)₅—CH₃
CH₃—CO—CH═CH—(CH₂)₄—CH₃
CH₃—CO—CH═CH—(CH₂)₃—CH₃
CH₃—CO—CH═CH—(CH₂)₂—CH₃ [Formula 4]

6. A microtubule-disrupting agent, comprising an α,β-unsaturated carbonyl compound having a chemical structure in common with 6-shogaol as an active ingredient.

7. The microtubule-disrupting agent according to claim 6, wherein the compound is a compound defined by the following chemical formula:
R₁—R₂—CO—CH═CH—R₃ [Formula 1]
wherein R₁ is an aryl group having 6 to 18 carbon atoms, is an alkyl group having 1 to 10 carbon atoms, and R₃ is an alkyl group having 1 to 10 carbon atoms.

8. The microtubule-disrupting agent according to claim 6, wherein the compound is a compound defined by the following chemical formula:
R₁—CO—CH═CH—R₂ [Formula 2]
wherein R₁ is an alkyl group having 1 to 10 carbon atoms, and R₂ is an alkyl group having 1 to 10 carbon atoms.

9. The microtubule-disrupting agent according to claim 6, wherein the compound is a compound defined by the following chemical formula (6-shogaol):

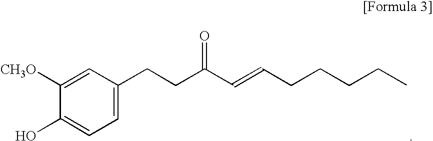

10. The microtubule-disrupting agent according to claim 6, wherein the compound is a compound defined by any one of the following chemical formulas:
CH₃—CO—CH═CH—(CH₂)₅—CH₃
CH₃—CO—CH═CH—(CH₂)₄—CH₃
CH₃—CO—CH═CH—(CH₂)₃—CH₃
CH₃—CO—CH═CH—(CH₂)₂—CH₃ [Formula 4]

11. A cancer cell proliferation inhibitor, comprising a microtubule-disrupting agent according to claim 6.

12. The cancer cell proliferation inhibitor according to claim 11, which is used for prevention or treatment of cancer.

13. The cancer cell proliferation inhibitor according to claim 12, wherein the cancer is selected from the group consisting of gastric cancer, acute T-cell leukemia, and colon cancer.

14. A use of a compound according to claims 6 for producing a cancer cell proliferation inhibitor.
